(19) United States
(12) Patent Application Publication
(10) Pub. No.: US 2007/0224242 A1
(43) Pub. Date: Sep. 27, 2007

(54) Tetrahedral Amorphous Carbon Coated Medical Devices

(75) Inventors: John Helmuth, Holt, MI (US); Hans-Joachim Scheibe, Dresden (DE); Thomas Schuelke, Brighton, MI (US); Gary L. Woodrough, Holt, MI (US)

Correspondence Address: DICKINSON WRIGHT PLLC, 38525 WOODWARD AVENUE, SUITE 2000, BLOOMFIELD HILLS, MI, 48304-2970, US

(73) Assignee: JET ENGINEERING, INC., Lansing, MI (US)

(21) Appl. No.: 11/688,655

(22) Filed: Mar. 20, 2007

Related U.S. Application Data
(60) Provisional application No. 60/743,614, filed on Mar. 21, 2006.
(60) Provisional application No. 60/785,942, filed on Mar. 24, 2006.

(51) Int. Cl. A61F 2/06

(52) U.S. Cl. 424/423; 623/1.11

(57) ABSTRACT

A medical device, such as an orthopedic or prosthetic joint, has a protective coating bonded to the substrate material of the device. Suitable substrate materials may include pure metals and metal alloys, ceramics, polymers and composites of the above. The protective coating includes a thin layer of tetrahedral bonded Carbon (ta-C). The coating also optionally includes an interface layer to facilitate the initial bonding and retention of the ta-C layer, such as by acting as an adhesion promoter, fluid barrier layer, or coefficient of thermal expansion mismatch reducing layer, or combination thereof. The interface layer may include various tightly adherent metals and metal nitrides, such as Cr, Ti, Nb, Ta and carbides, nitrides and carbonitrides thereof. The ta-C layer has a concentration of sp3 bonded carbon which varies through its thickness. Many concentration profiles of sp3 carbon bonds through the thickness are possible. The ta-C layer may also be doped with various materials, either through its thickness, or at either an inner or an outer interface, or both. The doping may include any suitable dopant, including various pure metals or metal alloys, bone growth substances, and dopants which alter the tribology of the ta-C layer with respect to the fluids in which it is in contact, such as dopants comprising F and N, or combinations of these materials.

### CROSS REFERENCE TO RELATED APPLICATIONS

This patent application claims priority to provisional patent application No. 60/743,614 filed Mar. 21, 2006 and 60/785,942 filed on Mar. 24, 2006, which are hereby incorporated by reference herein in their entirety.

### BACKGROUND OF THE INVENTION

1. Technical Field

This invention relates generally to medical devices having a carbon coating on an exterior surface thereof. More particularly, the invention relates to medical devices having a tetrahedral bonded amorphous carbon coating on a contact surface thereof. The contact surface may include wear surfaces, such as exist in various types of orthopedic joints, as well as fixation surfaces that are in contact with various bodily fluids or tissues.

2. Related Art

Various types of medical devices exist which are designed to have extended or permanent contact with human tissue and associated bodily fluids. These devices include various implants, including orthopedic and non-orthopedic implants. They also include various forms of surgical screws, pins, tubes, needles and other devices that may remain in contact with tissue and bodily fluids for extended periods of time. Surgical instruments such as scalpels, drills, reamers, bushings, saws, broaches and many other similar devices, as well as components of these devices, may also have relatively extended contact with tissue and bodily fluids. In the case of these devices, and particularly for those devices which are used as temporary or permanent implant devices, it is essential to control the exchange of materials of the device with the surrounding tissue and bodily fluids in which they are in intimate touching contact. In such cases, various types of coatings have been proposed to limit the exchange of the coating material and underlying substrate with the surrounding bodily fluids and/or tissues.

One such example is a diamond-like coating applied to an orthopedic implant as described in U.S. Pat. No. 6,626,949 B1 to Townley. In this patent, various types of joint implants are described which have a diamond or diamond-like coating applied to the concave wear surface of the joints. In particular, the application of such a coating to a wear surface of an acetabular cup made from a polymer of ultra high molecular weight polyethylene (UHMWPE) or polyurethane is described. This patent also describes the use of a diamond or a diamond-like coating on the mating convex wear surface of the joint, such as a metal or ceramic ball. The application of the diamond or diamond-like coating to the polymeric material also results in the creation of a polymeric or organic transition layer between the polymeric material of the substrate and the diamond or diamond-like coating which includes various carbon moities that are a result of the processes used to form the diamond or diamond-like film.

Townley is but one example of the application of a diamond or diamond-like coating on a medical device. These coatings have historically been made using a variety of processes. Characteristically, processes utilized for these depositions have involved either high deposition temperatures, or required that the substrate material be maintained at a relatively high temperature; however, high temperature processes to apply to the diamond or diamond-like materials which may be utilized for these applications, particularly polymeric materials, are undesirable as the elevated temperatures associated with their deposition can have deleterious effects on the structure and/or properties of the polymer.

The hardest known material is carbon in its crystalline form. In addition to its hardness, it is also resistant and substantially inert with respect to the substances which comprise human tissue and bodily fluids. Crystalline diamond films can be deposited by chemical vapor deposition (CVD) methods from a C-containing (CH₄ or C₂H₂)/hydrogen gas mixture. The deposition of crystalline diamond films using CVD requires high temperatures (e.g., >800° C.) on the surface of the material to be coated. Such films usually have a higher roughness than DLC and have to be polished before they can be used as wear protective coatings. This process makes such films undesirable for many commercial applications due to the high substrate temperatures and the necessity of the additional processing needed to polish the films.

A new alternative diamond film with purportedly better corrosion and wear characteristics for these application is nanocrystalline diamond. Nanocrystalline diamond (NCD) is deposited using plasma-assisted chemical vapor deposition (PECVD) to form thin films of diamond that consist of nano-sized crystals of diamond each a few to 10's of nanometers in size. The PECVD processes that are used generally consist of either a hydrogen-methane based process or an argon-methane based process. These nanocrystalline films can be grown to thicknesses ranging from less than 1 micron to 10's of microns, and they have been shown to be quite smooth and pin-hole free. A potential advantage of these films is that they can be grown essentially stress free, which can be advantageous in some coating applications where stress in the film can cause adhesion or other problems. The disadvantage of these films is that the PECVD process occurs at high temperatures that are often 600-900° C., but can be extended with some effort down to about 300-400° C.

By means of different CVD methods, amorphous diamond-like carbon (DLC) films can also be deposited at lower temperatures (about 200° C.) from a C-containing atmosphere. These films reach a maximum hardness of about 20 GPa, because the have a hydrogen content between about 5 and 50 atom percent. With increasing hydrogen content, the films become increasingly softer, until they reach polymer-like properties. Besides the hydrogen content, the film properties are mainly dominated by the ratio of diamond bonds (sp³) to graphite bonds (sp²) of the carbon atoms. Hydrogen containing amorphous carbon films have been called C—H films. The highest diamond-likeness (sp³) is actually reached in amorphous carbon films, containing virtually no hydrogen, using physical vapor deposition methods (PVD). These films are called tetrahedral bonded amorphous carbon films (ta-C) and are deposited from a highly activated pure carbon plasma. Such plasma is produced by pulse laser deposition (PLD) or pulsed arc discharge evaporation of pure graphite under vacuum conditions. The film deposition is mainly dominated by highly energetic carbon ions, which are able to penetrate into the surface of the material to be coated. There they form local Sp bonds, which are stabilized at a low temperature (below 100° C.). The deposition process is called subplantation and allows coating materials which are sensitive to chemical or physical alteration or decomposition by exposure to elevated temperatures, such as many polymer materials, with super hard carbon films.

Extensive development work has been carried out to develop the CVD and PVD methods for deposition of crystalline and amorphous carbon films with improved properties for different applications. The use of carbon coatings (crystalline or amorphous) for medical implants, surgical instruments and tools for production of pharmaceutical products have also been a focus of research because of their biocompatibility.

For example, CoCrMn alloys, a commonly used implant material for artificial joint stems, was coated with ta-C by using a filtered pulsed arc deposition method (FPAD). A film thickness from 1-200 μm could be deposited with an excellent adhesion on this material by a special interface preparation. The corrosion rate of a 1 μm thick coated substrate could be reduced by a factor of 10⁵ when it was exposed to a saline solution equivalent to bodily fluids at 37° C. for 2 years. The corrosion rate decreases with increasing thickness of the film. Diverse tribological tests (pin on disc and pin on plate) have demonstrate that the wear of ta-C coated metal joints is more than 10⁵ lower than in conventional metal—polyethylene and metal—metal pairs. The wear of the polyethylene shell is reduced at least 10 times if the metal ball of the joint is coated with ta-C. Tests with a hip joint simulator have shown that loads can be applied up to 1300 kg on ta-C coated joints without failure. Under a 200 kg load the coefficient of friction was 0.05, 0.05 and 0.14 for ta-C coated metal, metal—polyethylene and metal—metal pairs, respectively. The very low friction coefficient properties of the ta-C film will also help to reduce the torsional stresses that are usually accompanied with the early loosing of the stem in the bone. Animal studies have been carried out which indicate a benign biological response. In addition to only nominal wear, the amount of particles causing a foreign body reaction is 10⁵-10⁶ lower compared to metal on metal pairs. But, in contrast to the metal particles and metal ion contamination, the carbon particles do not cause tissue reactions or inhibit bone growth. In addition to decreased amounts of noxious compounds, this means a very low probability of detachment or delamination of the coating. Furthermore, in the case of orthopedic implants the carbon coating helps reduce the bone cement wear, which should improve the bone cement to implant bonding.

While ta-C coatings have shown great promise for application to medical devices, there exists a need to improve these coatings, to enable broader application and use of ta-C coatings with various medical devices.

### SUMMARY OF THE INVENTION

In one aspect, the invention is a device, such as a medical device which is adapted for contact with animal tissue or bodily fluids having a protective coating which includes a tetrahedral amorphous carbon layer, particularly wherein the composition or morphology, including the concentration of sp3 carbon bonds of the layer, varies through its thickness. The surface of the device acts as a substrate, and may include virtually any non-volatile solid substrate, including pure metals and metals alloys, ceramics, polymers and composites thereof. These materials may include metals; such as CoCr alloys, Ti and TiAl alloys, stainless steel alloys, high and low carbon steel alloys; polymers, such as UHMWPE, PEEK, PEK, polyphenylsulphone, polyurethane, and various other thermoset and thermoplastic materials, ceramics, such as Al₂O₃, Si₃N₄, TiN, SiC and other metal oxides, nitrides, carbides and combinations thereof, as well as composites of the materials listed above.

In another aspect, the protective coating also includes an interface layer acting to facilitate the initial bonding and subsequent retention of the ta-C layer, such as by acting as an adhesion promoting, fluid barrier layer, or coefficient of thermal expansion mismatch reducing layer, or combinations thereof. The interface layer may include various tightly adherent metals and metal nitrides, such as pure metals or alloys of Cr, Ti, Nb and Ta, and their carbides, nitrides and carbonitrides.

In another aspect, the ta-C layer has a sp3/(sp2+sp3) ratio that varies through the thickness of the layer. The sp3/(sp2+sp3) ratio may vary through the thickness according to virtually any desired profile, including various linear, curvilinear, step and other profiles, and combinations thereof.

In another aspect, the ta-C layer may also have a sp3/(sp2+sp3) ratio that varies through the thickness by being doped with various materials, either through its thickness, or at either an inner or an outer interface, or both. The dopant may include any suitable dopant, including various pure metals or metal alloys, various substances used to promote bone growth, and dopants which alter the tribology of the ta-C layer with respect to the fluid or fluids with which it is in contact, such as dopants comprising F and N, and combinations of these elements.

These and other features and advantages of this invention will become more apparent to those skilled in the art from the detailed description of a preferred embodiment. The drawings that accompany the detailed description are described below.

### BRIEF DESCRIPTION OF THE DRAWINGS

FIG. 1 is a partial section view of a ta-C coated hip joint device of the present invention;

FIG. 2a is a perspective view of a ta-C coated ball/stem of a hip joint device of the present invention;

FIG. 2b is a perspective view of a ta-C coated acetabular cup of a hip joint device of the present invention;

FIG. 3 is a partial section view of a ta-C coated shoulder joint device of the present invention;

FIG. 4 is a perspective view of a ta-C coated knee joint device of the present invention;

FIG. 5 is a top view of a portion of a ta-C coated elbow joint device of the present invention;

FIG. 6 is a perspective view of a portion of a ta-C coated spinal joint device of the present invention;

FIG. 7 is a perspective view of a ta-C coated bone screw device of the present invention;

FIG. 8 is a top view of a ta-C coated fixation device or surgical pin device of the present invention;

FIG. 9 is a schematic representation of the ta-C layers of the invention, as well as an optional interface layer; and

FIG. 10 is a schematic representation of the variation of concentration of sp3 carbon bonds through the thickness of a ta-C layer of the invention.

### DETAILED DESCRIPTION OF A PREFERRED EMBODIMENT

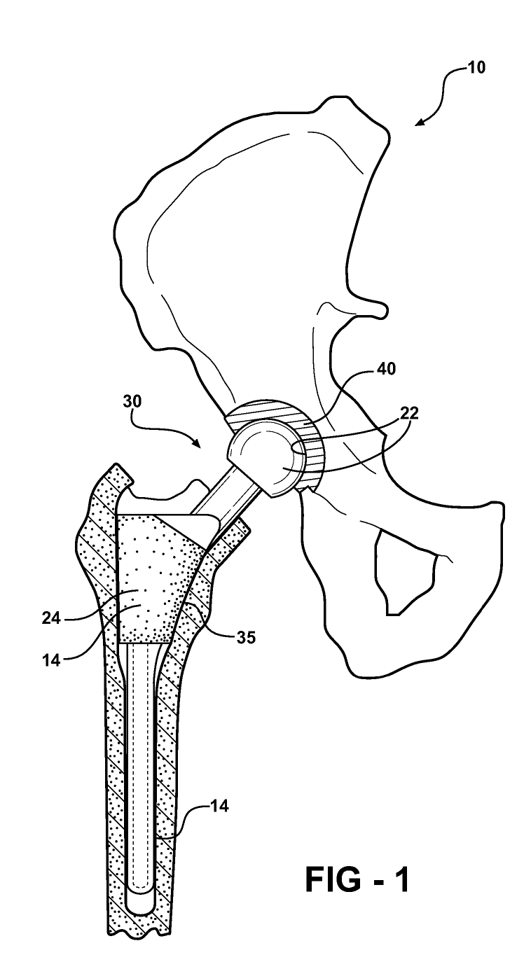

As shown in FIGS. 1-8, the present invention is a medical device 10 having a contact surface 12 which is adapted for intimate touching contact with animal tissue or bodily fluids, particularly human tissue and bodily fluids. On at least a portion of contact surface 12 there exists a protective coating 14 which comprises a thin layer or film of tetrahedral bonded amorphous carbon 16.

Medical device 10 may comprise any of a number of medical devices that are used in intimate touching contact with animal tissue or bodily fluid, such as human tissue or bodily fluids. Medical device 10 may include devices which are used for long-term or permanent contact with animal tissue and bodily fluids, including all manner of implantable devices, such as artificial joints and various orthopedic and prosthetic devices, which are totally or partially immersed in animal tissue and bodily fluids for relatively long periods of time ranging from, for example, several hours to many years. This also includes all manner of internal and external tissues and bodily fluids, including devices which have dermal and transdermal tissue contact, and includes perspiration as a bodily fluid, which is well known to be highly corrosive to many materials.

Medical device 10 may also include any of a number of medical instruments or surgical tools or other devices which are designed for relatively short-term contact with animal tissue or bodily fluids, typically up to several hours in duration.

Whether medical device 10 is intended for long-term or permanent contact with animal tissue and fluids or temporary or shorter periods of contact, it is generally desirable that the material comprising contact surface 12 be adapted to minimize, and preferably prevent, the migration of this material into the adjoining animal tissue and associated bodily fluids, whether by dissolution, corrosion or other chemical processes for causing such migration, or whether due to various wear, impact or other physical processes for causing the removal and consequent migration, such as by abrasion, ablation or other physical processes by which material may be removed from contact surface 12 and find its way into the tissue or fluids.

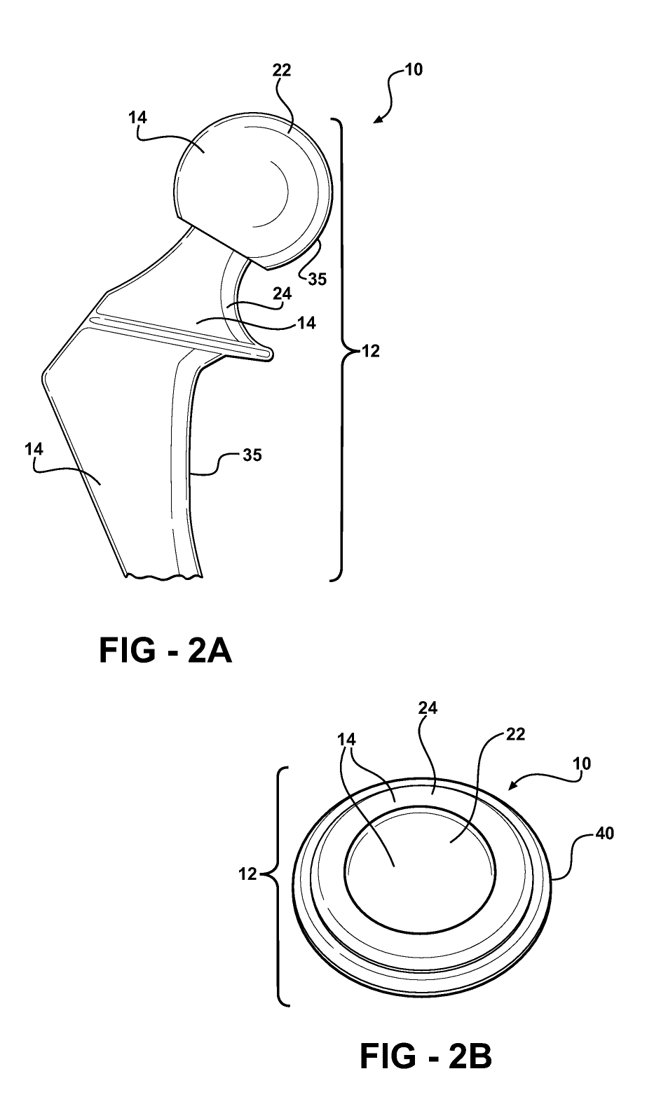

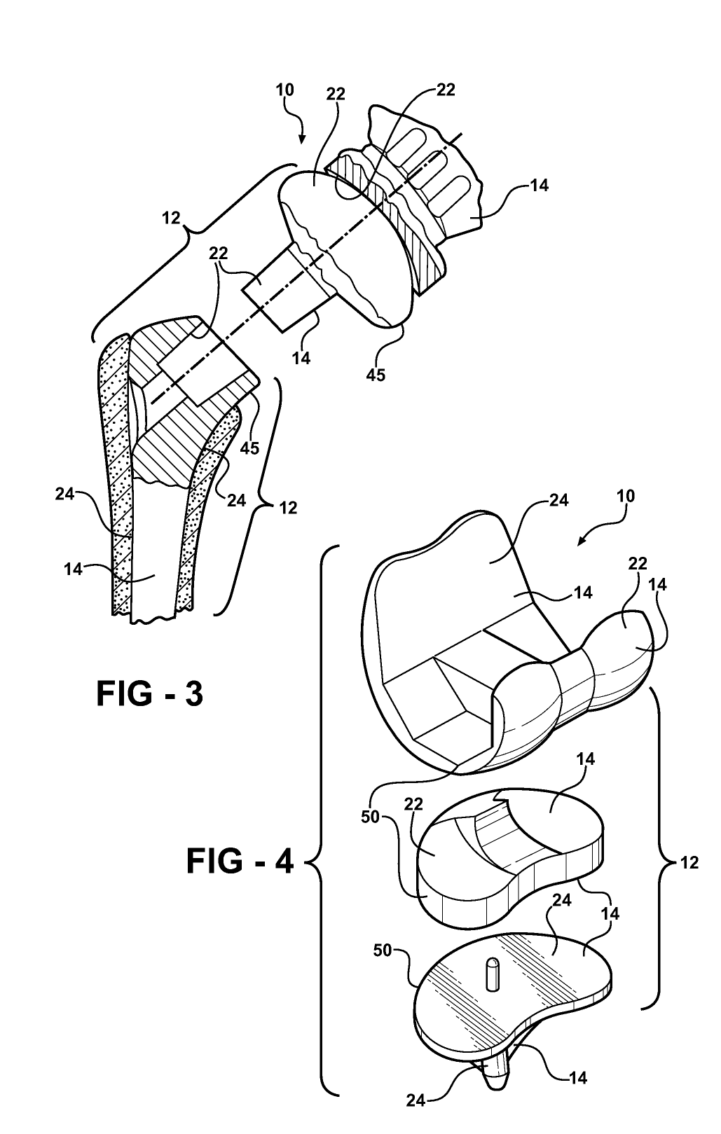

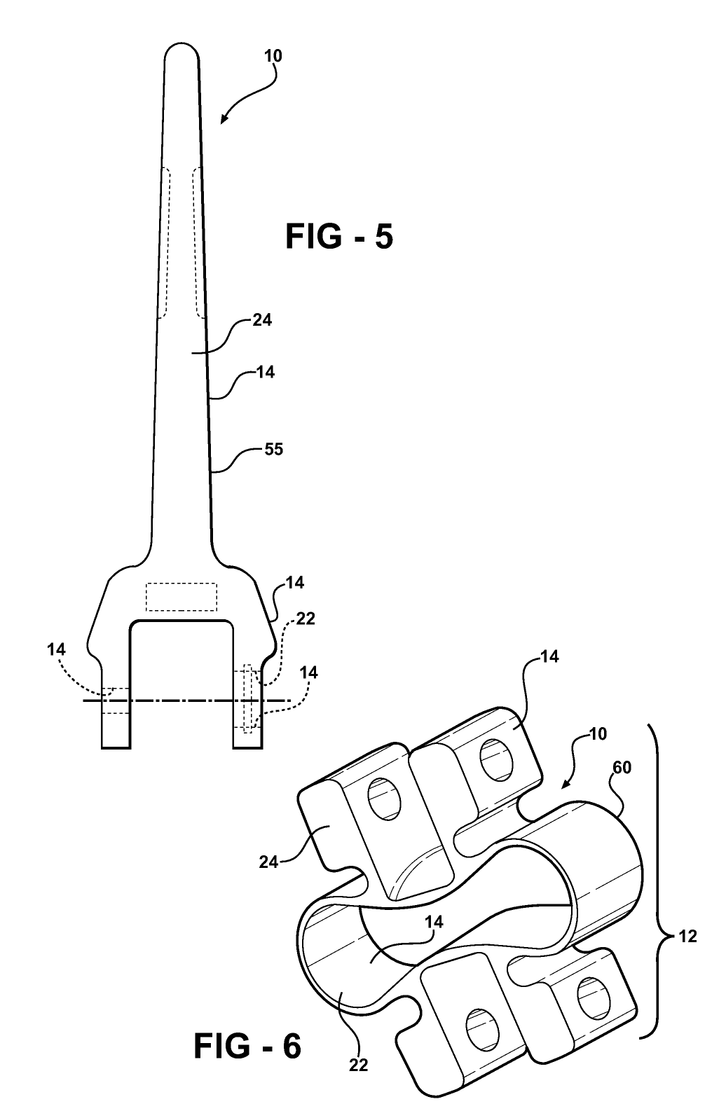

As shown in FIGS. 1-8, medical device 10 may include an orthopedic device in the form of an artificial joint, such as hip joint 30 (FIGS. 1, 2A and 2B), including ball/stem 35 (FIG. 2A) and acetabular cup 40 FIG. 2B, a shoulder joint 45 (FIG. 3); knee joint 50 (FIG. 4); elbow joint 55 (partially illustrated in FIG. 5); spinal joint 60 (FIG. 6), as well as numerous digital joints (not shown) of the hand and foot, and the like.

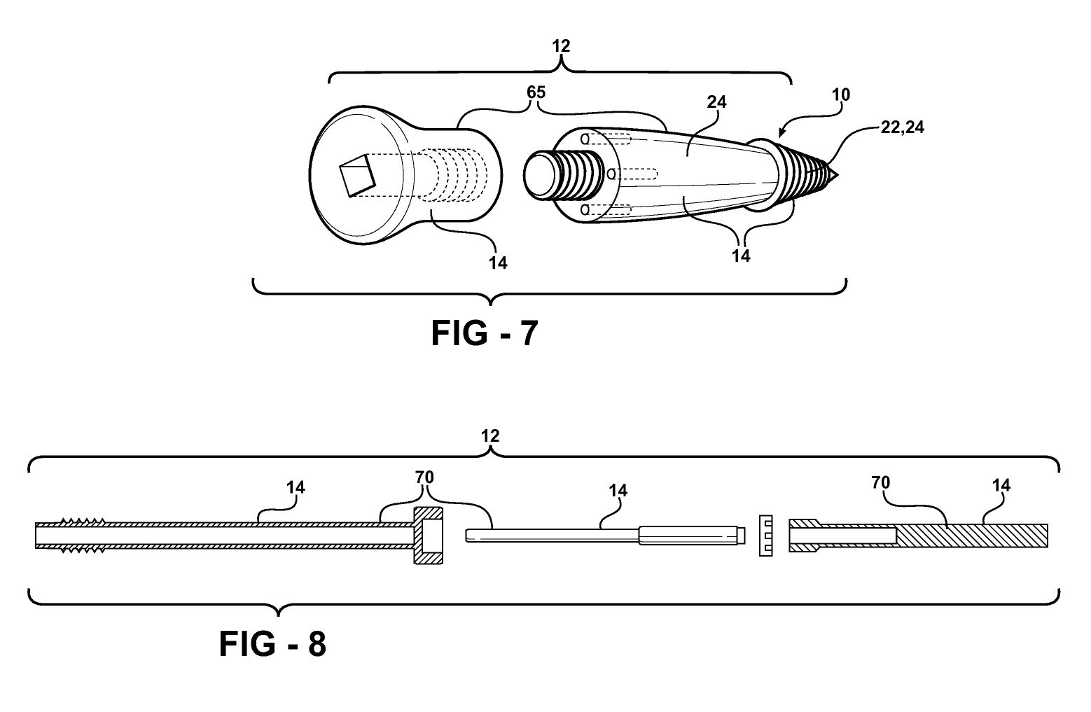

Medical device 10 may also include non-joint orthopedic implantable devices, such as various forms of temporary and permanent surgical fasteners, such as screw 65 (FIG. 7) or other fasteners, and various temporary or permanent pins, such as pin assembly 70 (FIG. 8). Medical device 10 may also include devices (not shown) such as stents, cardiac pacemakers, cardiac defibrillators, muscle stimulators, neurological stimulators, cochlear implants, monitoring devices, drug pumps and various other implantable non-orthopedic devices. Medical device may also include all manner of braces and prosthetic devices, such as artificial arms or legs, as well as anchors and attachment points for the same that have either temporary or permanent dermal, transdermal or subdermal contact. When medical device 10 comprises an implantable device, the device may be a permanently implanted device, or alternately, may be a device which is implanted only temporarily.

Medical device 10 may also include many non-implantable devices, including surgical instruments and tools, such as various reamers, including patellar reamers7 and acetabular reamers (not shown). Medical device 10 may also include various embodiments of surgical drills, scalpels, surgical bushings, surgical broachs, surgical rasps, surgical saw guides (not shown), and many other types and configurations of surgical instruments as medical device 10. Exemplary embodiments of the medical devices to which it is believed that the ta-C coating layer 14 of the present invention may be applied are illustrated and taught in FIGS. 1-8, and further explained in U.S. Pat. Nos. 4,822,364; 5,807,407; 6,203,576; 6,471,707; 6,537,274; 6,645,216; 6,770,077; 6,899,736; 6,932,821 and U.S. Pending Patent Application 2006/0015181, which are hereby incorporated herein by reference in their entirety. The medical device may also include components of the devices described and illustrated above.

As shown in FIGS. 1-9, medical device 10 has a contact surface 12 that is adapted for contact with animal tissue, bodily fluids or both, when the device is in use. Contact surface 12 may constitute a single contact surface 12 or multiple contact surfaces 12. Contact surface or surfaces 12 may have the same function or different functions depending upon the application of the medical device 10. For example, in the case of joint devices, contact surface 12 may comprise a wear surface 22. Contact surface 12 may also comprise a fixation surface 24 which is used to fix medical device 10 to tissue, such as bone or muscle tissue. Contact surface 12 may comprise the entire exterior surface of the device when medical device 12 is used as a temporary or permanent implant device, or any portion thereof.

Protective coating 14 may be used to coat all of contact surface 12 or only a portion or portions thereof. For example, when medical device 10 comprises an implantable joint, medical device 10 has a wear surface 22 and it is highly desirable to apply protective coating 14 over the entirety of the wear surface so as to improve the wear characteristics of the wear surfaces 22, such as reducing the friction coefficient and avoid abrasion or other degradation of the contact surface 12 of the underlying substrate 19 material. While it is desirable to coat the wear surfaces 22 of a medical device such as a joint, it may, depending on the application, be desirable to leave the remaining portion or portions of contact surface 12 uncoated. It may also be desirable to coat the entire contact surface 12 with protective coating 14. Still further, it may be desirable to coat the wear surface of contact surface with a protective coating 14 having a first set of characteristics while applying protective coating 14 to other portions of contact surface 12, such as fixation surface 24, with a second and different physical or morphological or compositional characteristics, as explained further below.

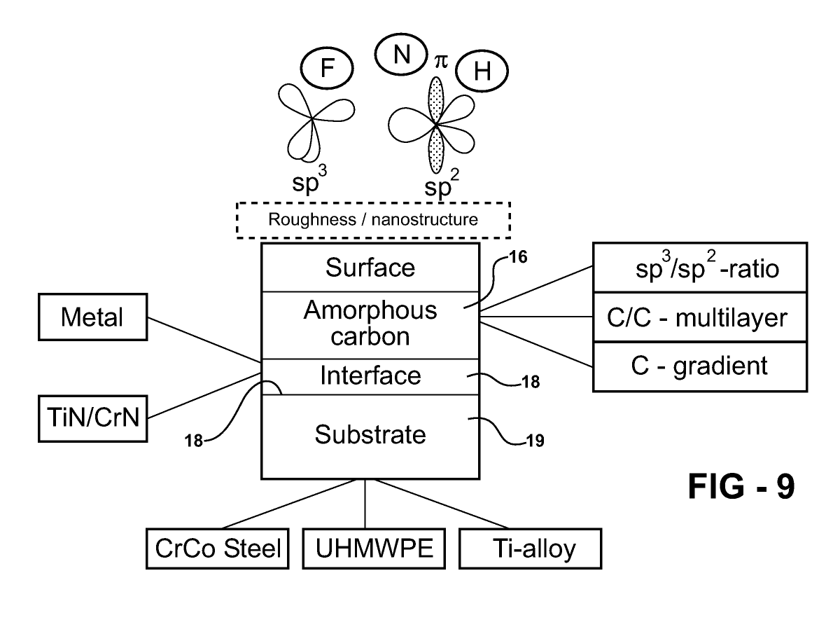

Protective coating 14 comprises tetrahedral amorphous carbon (ta-C) layer 16 applied to a portion of contact surface 12 as a substrate 19 as illustrated in FIG. 9. Optionally, an interface layer 18 is located between the contact surface 12 as the substrate and ta-C layer 16. The interface layer 18 may perform several functions depending on the substrate selected and the nature of the ta-C layer as explained further herein, including service as an adhesion promoter, a fluid or diffusion barrier layer, and a coefficient of thermal expansion (CTE) adjustment layer (e.g., to adjust for a CTE mismatch between the substrate 19 and ta-C layer 16). Interface layer 18 may be used as an adhesion promoter to improve the tensile or sheer strength of the bond between the ta-C layer 16 and the contact surface 12 of the substrate 19 over that which would occur if the ta-C layer were bonded directly to contact surface 12 material. The interface layer 18 may also serve as a fluid barrier layer in the case where bodily fluids may penetrate the ta-C layer in a way which is acceptable for the intended application, but which might otherwise provide a path for bodily fluids to reach the contact surface 12 of substrate 19 and allow exchange of the material of the substrate 19 with the bodily fluids. The interface layer 18 may also act as a barrier layer to the extent that ta-C layer 16 has a pattern (not shown) such that it is discontinuous (e.g. a dimpled pattern or pattern of raised ta-C layer 16 features with adjoining areas which do not include ta-C layer 16) or varies in thickness over its surface and would otherwise allow a path for bodily fluids to directly reach the contact surface 12 of substrate 19 or increase the likelihood of them reaching this surface. The interface layer 18 may also act as a CTE transition layer to the extent that there is a mismatch between the CTE of the substrate 19 and the CTE of the ta-C layer 16, by selecting a material for use as the interface layer which has a CTE intermediate between that of the aforementioned layers, or which is deposited in such a way as to provide a CTE gradient between these layers. The interface layer 18 may also act as an elastic modulus transition layer to the extent there is a mismatch between the elastic modulus of substrate 19 and ta-C layer 16, by selecting a material for use as the interface layer which has an elastic modulus between that of the aforementioned layers, or which is deposited in such a way as to provide an elastic modulus gradient between these layers. The interface layer 18 may have any suitable thickness depending on the intended function or functions described above, but generally is expected to be between 0.5 and 2 μm thick.

The substrate 19 material is dependent on the medical device 10 selected and may include a wide range of materials, including metals, polymers, ceramics and combinations and composites of them. The metals and metal alloys commonly used as substrate material will include Ti alloys and TiAl alloys, CoCr alloys, stainless steels, such as types 316, 420, 440, 455 and 17-4, carbon steels, including high carbon and low carbon steels and the like. The Ti alloys include pure titanium and alpha/beta type Ti alloys, such as Ti-6A1-4V (Wt. %), Ti-15Mo (Wt. %), Ti-35Nb-7Zr-5Ta (Wt. %), Ti-3Al-2.5V (Wt. %) and Ti—Al—Nb alloys, such as Ti-10.5Al-3.6Nb (At. %), Ti-21Al-29Nb (At. %), and Ti-15Al-33Nb (At. %). The CoCr alloys may also include CoCrMo alloys. The stainless steels may include types 316, 420, 440, 455 and 17-4, but many other stainless steels grades are also applicable for use as the substrate material. Various high carbon and low carbon steel grades may also be used as the substrate material, depending on the application.

However, the steels are generally preferred for non-implant devices. Implant devices such as orthopedic joints have frequently used CoCr alloys which have known disadvantages due to the fact that wear of these alloys produces metal ion concentrations of Co and Cr that have been detected in serum and urine and which are also known to have certain cytotoxic effects, including tissue necrosis, which sometimes necessitates the removal and replacement of implanted devices. TiAl alloys are favored, particularly because of the weight reduction which they offer, however, they are known to be difficult to coat with carbon coatings using prior art carbon coatings and coating processes, due to the high temperatures to which the alloys must be exposed using these deposition processes.

Substrates 19 may also include various polymers for use in orthopedic joints, and other medical devices 10, catheters and as components of various medical instruments. In these applications, it is desirable to use a protective layer of ta-C for a variety of reasons including increased wear resistance, chemical inertness and others. Polymers which may be utilized as a substrate material may include ultra high molecular weight polyethylene (UHMWPE), polyetheretherketone (PEEK), polyetherketone (PEK), polyphenylsulphone, polyurethane and various other thermoset and thermoplastic materials. The present invention is particularly advantageous for use in conjunction with implantable orthopedic devices, such as the acetabular cup for a ball-cup type joint, such as a hip joint or shoulder joint. The use of uncoated UHMWPE is limited because of a wear rate on the order of 30-100 mm³/million cycles. In addition to the wear rate which is one to two orders of magnitude greater than the wear rate of a CoCr alloy in the same application, the polymeric wear debris resulting from the higher wear rate may also have undesirable cytotoxic effects. The application of a protective coating layer 14 to these devices is believed to offer a substantial reduction in the wear rate, on the order of an order of magnitude reduction. The incorporation of the interface 18 layer is also particularly advantageous in that it offers the opportunity to improve the adhesion of the ta-C layer 16 to the substrate 19. The lowered wear rate is also expected to have a significant advantage in lowering the cytotoxic effects associated with wear debris resulting from the wear of these materials. Various ceramics may also be used as a substrate material, including various metal oxide, metal nitride and metal carbide ceramics. While the use of these materials as substrate materials is somewhat limited at present, the ta-C layers of the present invention are equally applicable to these materials as they find increased use in various substrate 19 applications.

Various composite materials including metal matrix composites, ceramic composites and various polymer and polymer fiber and glass fiber based composites may also be used as substrate 19 materials. Again, while the use of these materials as substrates is currently limited, as they find increased applications as substrates 19 as described herein, the ta-C layers 16 of the present invention are equally applicable to these materials and in fact may provide even more benefit with regard to the application of these materials due to the enhanced chemical inertness of the ta-C layers 16 with regard to interaction between bodily fluids and tissues and the substrate 19 materials, particularly where the substrate 19 materials may include polymer resins as the composite matrix material.

The contact surface 12 may take many different forms depending upon the nature of the substrate 19 material and the device in which it is being used. In the case of the wear surface 22 of a device 10, such as an orthopedic implant, contact surface 12 is typically a highly-polished surface having a surface roughness of Ra<0.1 μm. The polishing may be performed using known polishing methods and techniques. This highly polished surface is a smooth continuous surface with no discontinuities in the surface. However, the enhanced wear resistance and chemical inertness provided by the ta-C layer 16 may offer the opportunity to alter such wear surfaces 22, such as by dimpling analogous to the patterns utilized in a golf ball cover, in order to reduce the contact area at the wear surface 22 and thereby reduce the frictional resistance to movement of the joint in-situ. For example, utilization of a dimple pattern where only the peaks of the pattern are polished to provide the bearing surface of the joint. This is facilitated by a ta-C layer 16 and potentially the interface layer 18, because of the enhanced hardness and wear resistance and reduced friction coefficient of the ta-C layer 16 as compared with that of the substrate 19 materials typically used in such devices previously, such as Co—Cr alloys and Ti—Al alloys. Further, the interface layer 19 is expected to further enhance the strength of the bond between the ta-C layer 16 and the substrate and offset the potential changes in the stress state of the ta-C layer 16 resulting from the patterning of the substrate 19 surface, such as increased contact pressures, shear forces and the like. Textured or patterned contact surfaces 12 may offer the benefit of distributing lubricant bodily fluids more evenly over the wear surface of the device. With regard to the non-wear or fixation surfaces 24 of the device, the ta-C layers of the present invention may be utilized with any of a number of known techniques for enhancing the fixation characteristics of the device in-situ, such as by providing porosity or enhanced surface roughness over portions of the fixation surface. In addition, the ta-C layers of the present invention may also be doped with materials that are known to promote tissue growth, as described herein. Further, doping may be used with ta-C layer incorporated over the wear surfaces 22, to further alter and enhance the tribological characteristics of the joint and the wear surface 22, such as by making the surface hydrophobic or hydrophilic.

Prior to the application of the interface layer 18 or ta-C layer 16 to the contact surface of the substrate, it is desirable to clean the contact surface either ex-situ or in-situ, with reference to the deposition apparatus utilized to apply these layers. Ex-situ substrate cleaning may be performed using known cleaning chemistries, ultrasonic cleaning, various heating cycles and even laser cleaning of the surface. In-situ substrate cleaning may be performed in connection with the application of the ta-C layer 16, or when the interface layer 18 is incorporated, prior to application of the interface layer 18. Any suitable technique may be utilized for in-situ cleaning, including reactive plasma etching, inert plasma sputter cleaning, radiant heating, metal ion plasma cleaning, carbon ion plasma cleaning and the like.

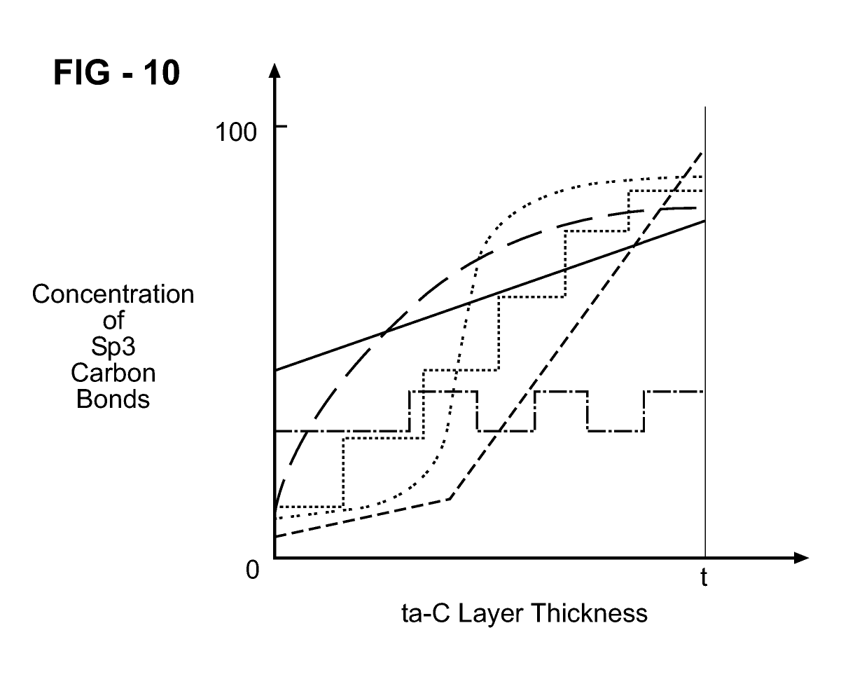

Generally, ta-C layer 16 of the invention will have a thickness depending on the thickness of interface layer 18 so that the combined thickness of both layers exceeds 5 μm on a metal substrate and 2 Mm on a UHWMPE substrate. Thicker ta-C layers 16 are possible and may be made using the deposition methods described herein with a high concentration of sp3 carbon bonds (e.g., up to about 70% sp3 bonds). However, while thicker layers 16 are possible, they are not necessarily more effective in providing the benefits associated with ta-C layers 16, as described herein. Therefore, cost considerations and the competitive marketplace generally favor the use of the thinner layer thicknesses in the range described herein. Applicants have observed that while thicker ta-C layers generally have acceptable strength and impact resistance and adhesion to substrates 19 or an interface layer 18 for use in the medical devices 10 described herein, this is sometimes not the case with thinner ta-C layers having thicknesses less than 2 μm. It is believed that this is due in part to the fact that the thicker layers have a thickness that is sufficient to distribute typical operating stresses in these devices within the layers themselves without developing stress concentrations at the contact surface 12 or outer surface of the interface layer 18 sufficient to cause separation of the layer 16 from these surfaces. However, this is not the case with layers having thicknesses less than about 5 μm. In such layers having uniformly high concentration of sp3 carbon bonds through the thicknesses of the layer, adherence of the layer may not be acceptable for medical devices 10, particularly those which are subject to relatively higher operating contact pressures, including various types of impact forces, such as various of the joints described herein. Therefore, Applicants have developed ta-C layers 16 which have variation in the concentration of sp3 carbon bonds through the thickness of the layer. The mechanical and other properties of ta-C layer 16 are a function of the degree or concentration of sp3 (diamond) and sp2 (graphitic) bonded carbon atoms. Since for pure ta-C layers 16 (i.e., without significant amounts of impurity or dopant atoms or compounds) deposited in accordance with this invention, essentially all of the carbon atoms have sp2 or sp3 bonds with a relatively higher concentration of sp3 bonds, the sp3/(sp2+sp3) ratio is a useful means for expressing this concentration. As the concentration of sp3 bonded carbon atoms increases, the hardness of ta-C layer 16 increases as well as the elastic modulus. By varying the concentration of sp3 carbon bonds and the sp3/(sp2+sp3) ratio through the thickness of these layers it is possible to more closely match the mechanical and physical properties of the ta-C layer to that of the substrate 19 or intermediate layer 18 and improve the adherence of ta-C layer 16 to these layers, thereby enabling the use of thinner ta-C layers 16 in the thickness range described herein. While this invention is enabling of thinner ta-C layers 16, it is also applicable for use with thicker ta-C layers and expected to provide similar benefits when applied thereto. The variation of the sp3 concentration through the layer thickness may occur according to any suitable concentration profile, such as those illustrated schematically in FIG. 10. Generally, the sp3 concentration at the contact surface 12 of substrate 19, or optionally the outer surface of interface layer 18, will be selected to closely match one or more properties of this material, such as the elastic modulus. The sp3 concentration at the free surface will generally be selected to optimize the performance of the ta-C layer, such as its hardness. In many cases this sp3 concentration will be about the upper concentration limit for commercially available ta-C films which is about 70% atom percent for essentially pure carbon films (i.e., without dopants). Generally the concentration profile will be selected to increase through the layer thickness to the free surface as shown in FIG. 10, but depending on the application this need not be the case and the reverse is possible, as well as concentration profiles which exhibit one or more maxima or minima on the interior of the layer away from either surfaces (not shown).

The sp3 concentration may be varied through the thickness of the layer using any suitable technique or combination of techniques in conjunction with known methods of depositing ta-C films. In one example, control of the sp3 concentration may be affected by controlling the temperature of the deposition surface of the substrate or interface layer, either by cooling or heating. While a threshold localized (on an atomic scale) temperature and pressure are necessary to achieve subplantation and the creation of sp3 carbon bonds, generally higher deposition temperatures will tend to produce a lower concentration of sp3 carbon bonds and corresponding higher concentration of sp2 carbon bond. In another example, the angle of incidence of the carbon atoms on the substrate may also be varied during deposition to alter the sp3/sp2 concentrations and achieve the profiles described herein, either by changing the position of the substrates during deposition using planetary and other fixtures for changing the substrate position, or by altering the position of the deposition source relative to the substrate targets. Alternately, the introduction of an inert gas (e.g., argon and other inert gases) or a reactive gas (e.g., nitrogen) may be used to scatter the carbon atoms during deposition and alter the angle of incidence, thereby changing the sp3/sp2 concentration in the deposited films. Substantially normal incident angles of the carbon atoms with the substrate are needed to produce sp3 bonded carbon films. Other angles of incidence will tend to increase the concentration of sp2 bonded carbon atoms in the deposited film.

The ta-C layers 16 of the invention will generally have a concentration of sp3 carbon bonds sufficient to produce an average elastic modulus through the thickness of the layer of at least 400 GPa. Typically it is desired to have a higher than average elastic modulus on the outer or free surface of the ta-C layer. Elastic moduli of up to about 700 GPa have been observed in ta-C films having the highest concentration of sp3 carbon bonds.

As shown by the stepped and square wave profiles in FIG. 10, the ta-C layer 16 of the invention having a varying concentration of sp3 carbon bonds through the thickness of the layer may also include a plurality of ta-C layers having at least two different constant sp3 concentrations. In the case of a stepped profile, this may include any number of different constant concentrations of sp3 carbon bonds.

As noted above, the ta-C layer 16 may also be doped, either through the thickness or at either of the inner or outer surfaces, such as by the introduction of reactive species such as N or F. This of course will also be expected to change the concentration or degree of sp3 carbon bonds in the ta-C layers 16. The profiles described above are also believed to be achievable when doping ta-C layers 16, although the profiles may shift due to changes in the concentration of sp3 carbon bonds. It is believed that many pure metals and metal alloys may also be used as dopants. Other dopants are also believed to be possible including dopants which act an adhesion promoters, tissue growth promoters, tribology modifiers and a coefficient of thermal expansion modifiers. For example the incorporation of aluminum as well as fluorine will increase hydrophobicity, which may help to prevent cell attachment. The corresponding reduction in hardness will be less for aluminum dopants than for fluorine. The incorporation of silver is believed to increase compatibility with the human body.

The ta-C layers 16 of the present invention may be deposited using the processes described in U.S. Pat. Nos. 6,231,956; 6,410,125; 6,338,778 and 6,533,908, which are hereby incorporated herein by reference in their entirety. The interface layers 18 may be made using known deposition techniques.

In addition to medical device 10, the ta-C layers 16 of the present invention may also be applied and provide similar benefits to a wide variety of non-medical devices which are formed from the materials described herein as substrate 19 materials for medical device 10.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

We claim:

1. A medical device, comprising:
a substrate having a contact surface; and
a ta-C layer bonded to said contact surface having a varying concentration of sp3 carbon bonding through a thickness of said layer.

2. The medical device of claim 1, wherein said ta-C layer has an average elastic modulus of at least 400 GPa.

3. The medical device of claim 1, wherein said concentration of sp3 carbon bonding comprises with reference to said contact surface one of an increasing or a decreasing concentration gradient through said thickness.

4. The medical device of claim 1, wherein said ta-C layer comprises a plurality of ta-C layers.

5. The medical device of claim 4, wherein said plurality of ta-C layers comprises an arrangement of layers having at least two different constant concentrations of sp3 carbon bonding.

6. The medical device of claim 1, wherein said ta-C layer further comprises a dopant.

7. The medical device of claim 7, wherein said dopant comprises at least one of an adhesion promoter, a tissue growth promoter, a tribology modifier and a coefficient of thermal expansion modifier.

8. The medical device of claim 1, wherein said substrate comprises a material selected from a group consisting of pure metals, metal alloys, ceramics, polymers and composites thereof.

9. The medical device of claim 8, wherein said substrate is a metal alloy selected from a group consisting of CoCr alloys, TiAl alloys, stainless steel alloys, high carbon steel alloys and low carbon steel alloys.

10. A medical device, comprising:
a substrate having a contact surface, wherein the contact surface is adapted for contact with animal tissue or bodily fluids;
an interface layer bonded to said contact surface; and
a ta-C layer bonded to said interface layer having a varying concentration of sp3 carbon bonding through a thickness of said layer.

11. The medical device of claim 10, wherein said ta-C layer has an average elastic modulus of at least 400 GPa.

12. The medical device of claim 10, wherein said concentration of sp3 carbon bonding comprises with reference to said interface layer one of an increasing or a decreasing gradient through said thickness.

13. The medical device of claim 10, wherein said ta-C layer comprises a plurality of ta-C layers.

14. The medical device of claim 13, said plurality of ta-C layers comprises an arrangement of layers having at least two different constant concentrations of sp3 carbon bonding.

15. The medical device of claim 10, wherein said ta-C layer further comprises a dopant.

16. The medical device of claim 15, wherein the dopant comprises at least one of an adhesion promoter, a tissue growth promoter, a tribology modifier, and a coefficient of thermal expansion modifier.

17. The medical device of claim 10, wherein said substrate comprises a material selected from a group consisting of pure metals, metal alloys, ceramics, polymers and composites thereof.

18. The medical device of claim 10, wherein said substrate is a metal alloy selected from a group consisting of CoCr alloys, TiAl alloys, stainless steel alloys, high carbon steel alloys and low carbon steel alloys.

19. The medical device of claim 10, wherein said interface layer comprises at least one of a metal, a metal nitride, a metal carbide and a metal carbonitride.

20. The medical device of claim 10, wherein said interface layer is a metal selected from a group consisting of Cr, Ti, Nb and Ta.
